(19) United States
(12) Patent Application Publication
(10) Pub. No.: US 2024/0165050 A1
(43) Pub. Date: May 23, 2024

(54) METHOD FOR TREATING AND/OR PREVENTING EDEMATOUS FIBROSCLEROTIC PANNICULOPATHY

(71) Applicant: CALIWAY BIOPHARMACEUTICALS CO., LTD., New Taipei City (TW)

(72) Inventor: YU-FANG LING, New Taipei City (TW)

(21) Appl. No.: 18/466,003

(22) Filed: Sep. 13, 2023

Related U.S. Application Data
(60) Provisional application No. 63/384,605, filed on Nov. 22, 2022.

(51) Int. Cl. A61K 31/12; A61K 9/00; A61K 9/107; A61K 31/05; A61P 17/00

(52) U.S. Cl. CPC A61K 31/12; A61K 9/0019; A61K 9/1075; A61K 31/05; A61P 17/00; B82Y 5/00

(57) ABSTRACT

The present disclosure provides a method for treating and/or preventing edematous fibrosclerotic panniculopathy (EFP) in a subject in need thereof. The method includes administering to the subject and effective amount of a pharmaceutical composition. The pharmaceutical composition includes a plurality of amphiphilic nanoparticles having one or more active ingredients encapsulated therein. Each of the amphiphilic nanoparticles is formed by a non-ionic surfactant, a polymeric carrier, or a lipid carrier. The hydrophilic-lipophilic balance (HLB) value of the non-ionic surfactant is greater than 9. The pharmaceutical composition is administered via a parenteral route by an injection, a microneedle, or an implant, or via topical administration or transdermal administration.

### CROSS-REFERENCE TO RELATED APPLICATION

The present application claims the benefit of U.S. provisional patent application No. 63/384,605, filed on Nov. 22, 2022, the entirety of which is incorporated herein by reference.

### FIELD OF THE INVENTION

The present disclosure relates to compositions for treatment or prevention of edematous fibrosclerotic panniculopathy (EFP). More specifically, the present disclosure relates to pharmaceutical compositions having drug-encapsulating micelles for reducing EFP or inhibiting EFP formation in a subject in need thereof.

### BACKGROUND OF THE INVENTION

Edematous fibrosclerotic panniculopathy (EFP), also known as cellulite, gynoid lipodystrophy, or local lipodystrophy, is a topographic alteration of the skin and subcutaneous adipose commonly observed on the buttocks, lower limbs, and abdomen. Etiopathogenesis of EFP involves expansion of subcutaneous fat, fibrotic dermal septa, dermal laxity and atrophy, excessive hydrophilic intracellular matrix, and microangiopathy and inflammation of the connective tissue. These changes eventually make the skin of the buttocks, lower limbs, and abdomen bumpy and irregular, thus resembling the look of “orange peels.” Although not life threatening, EFP remains an issue of cosmetic concern to a considerable number of individuals, especially to over 80% of postpubertal women around the world.

Mostly, EFP has negative impacts on women's psychosocial sphere because it is directly and inexorably related to the physical appearance, self-esteem, and consequently to the well-being perception and social acceptance. EFP can cause the body an unhealthy appearance, and reduce one's self-confidence. According to a Comprehensive Research Report by Market Research Future (MRFR), “Cellulite Treatment Market Information by Cellulite Type, End Use and Treatment Procedure—Forecast till 2027,” the market has a compound annual growth rate (CAGR) of 11.2% and is projected to reach $3,162.1 million USD by 2027. The market analysis demonstrates the increasing demand for EFP treatments.

The exact cause of EFP formation remains a controversial issue, but it appears to result from an interaction between the connective tissue in the dermis layer that lies below the surface of the skin, and the layer of fat that lies just below it. EFP is more common in females because fat cells and connective tissue in the dermatological layers are arranged vertically and fiber distribution therebetween is loose, which facilitates protrusion of fats and results in the appearance of EFP. EFP is often categorized based on its severity: (i) Grade 0: no EFP; (ii) Grade 1: smooth skin when standing, but orange-peel appearance when sitting; (iii) Grade 2: skin has orange-peel appearance when standing and sitting; and (iv) Grade 3: skin has orange-peel appearance when standing with deep raised and depressed areas. Over the past few decades, various non-invasive or invasive approaches to remove EFP have been provided.

For example, applying massage cream is a commonly used non-invasive approach. However, since EFP is caused by uneven accumulation of fat cells and existing massage creams only acts on the epidermis, it is almost impossible for the therapeutic ingredients to reach deeper into the subcutaneous tissue. Therefore, such method has failed to effectively eliminate EFP.

On the other hand, invasive approaches include liposuction, radiofrequency (RF) therapy, and laser therapy. Since EFP mainly occurs in the superficial fat and dermis, using liposuction to treat EFP will not be effective because in addition to the side effects, scar tissue and uneven skin will also be created. Radiofrequency (RF) therapies uniformly heat the entire area of the skin (through volume heating), which also act on the dermis and fat layer of the skin to achieve skin firmness and wrinkle removal. However, the risk of skin burn is high in RF therapies, and heat applied to the patients has to be carefully controlled throughout the procedure. In laser therapies, local anesthesia, formation of incisions, and catheter intrusion are required. Specifically, laser liposuction involves performing local anesthesia on the target body part and forming a small incision to extend a catheter under the skin, followed by using a laser probe to generate a low-energy laser beam to break down and remove subcutaneous. Other common treatments include acoustic wave therapy (AWT) and injectable treatments. However, most of the existing treatments are costly and often come with undesirable side effects, such as inflammation, redness, soreness, and bruising. Moreover, none of the procedures have been shown to be sustainable over time. Existing topical agents, injectable treatments, and energy-based approaches can only ameliorate the appearance of EFP, but are unable to eliminate or prevent recurrent of EFP.

Therefore, there is still a need in the art for a safer and more sustainable approach for treating and/or preventing EFP.

### SUMMARY OF THE INVENTION

In view of the deficiency of the prior art, the present disclosure provides pharmaceutical compositions and methods for treating and/or preventing EFP by using the same. The pharmaceutical compositions have the advantages of high stability, high bioavailability for fat tissues, minimal side effects, and sustained release. A method for treating and/or preventing EFP in a subject in need thereof is provided. The method includes administering an effective amount of a pharmaceutical composition to the subject. The pharmaceutical composition includes a plurality of amphiphilic nanoparticles having one or more active ingredients encapsulated therein. Each of the amphiphilic nanoparticles is formed by a non-ionic surfactant, a polymeric carrier, or a lipid carrier. The hydrophilic-lipophilic balance (HLB) value of the non-ionic surfactant is greater than 9.

Preferably, the amphiphilic nanoparticles are a plurality of micelles or emulsions encapsulating the one or more active ingredients and formed by the non-ionic surfactant.

Preferably, the amphiphilic nanoparticles are a plurality of polymeric nanospheres or polymeric nanocapsules encapsulating the one or more active ingredients and formed by the polymeric carrier.

Preferably, the amphiphilic nanoparticles are a plurality of liposomes encapsulating the one or more active ingredients and formed by the lipid carrier.

Preferably, each of the amphiphilic nanoparticles includes a hydrophobic core and a hydrophilic envelope. The one or more active ingredients is encapsulated in the hydrophobic core, and the hydrophobic core is encapsulated in the hydrophilic envelope. The hydrophobic core is formed by hydrophobic end groups of the non-ionic surfactant, the polymeric carrier, or the lipid carrier. The hydrophilic envelope is formed by hydrophilic head groups of the non-ionic surfactant, the polymeric carrier, or the lipid carrier.

Preferably, each of the amphiphilic nanoparticles includes a hydrophilic core and a hydrophobic envelope. The one or more active ingredients is encapsulated in the hydrophilic core, and the hydrophilic core is encapsulated in the hydrophobic envelope. The hydrophilic core is formed by hydrophilic head groups of the non-ionic surfactant, the polymeric carrier, or the lipid carrier. The hydrophobic envelope is formed by hydrophobic end groups of the non-ionic surfactant, the polymeric carrier, or the lipid carrier.

Preferably, the pharmaceutical composition is administered in a non-oral dosage form, such as a dosage form administered via a parenteral route by an injection, a microneedle, or an implant, or a dosage form for topical administration or transdermal administration. The dosage form administered by injection includes but not limit to, powder injection, lyophilized injection, sterilized suspension, injectable solution, injectable emulsion, and intravenous fluid; and the dosage form for topical or transdermal administration includes but not limit to, ointment, lotion, liniment, cream, gel, dressing, emulsion, film, patch, poultice, cataplasm, topical powder, and topical solution.

Preferably, the pharmaceutical composition is administered to thighs, buttocks, lower limbs, pelvic region, or abdomen of the subject.

Preferably, the pharmaceutical composition is administered to a treatment area of the human by subject. The effective unit dose injected to the treatment area is 0.01-50 mg/cm² of the treatment area. The pharmaceutical composition is administered at least once.

Preferably, the pharmaceutical composition is administered to a treatment area of the subject by the microneedle or the implant, and the effective unit dose injected to the treatment area is 0.01-20 mg/cm² of the treatment area.

Preferably, the pharmaceutical composition is administered to a treatment area of the subject by topical application, and the effective amount applied onto the treatment area is 0.5-20 mg/cm² of the treatment area.

Preferably, the pharmaceutical composition is administered to a treatment area of the subject by transdermal administration, and the effective amount applied onto the treatment area is 0.5-50 mg/cm² of the treatment area.

Preferably, the effective amount of the pharmaceutical composition is 40-80 mg. Preferably, the pharmaceutical composition is administered at least once.

Preferably, the effective amount is an amount sufficient to achieve at least one of the following efficacy endpoints: (a) to reduce a depth of a treatment area by at least 10%; (b) to reduce a width of the treatment area by at least 10%; (c) to reduce a length of the treatment area by at least 5%; (d) to reduce an overall volume of the treatment area by at least 10%; and (e) to reduce a surface area of the treatment area by at least 10%.

Preferably, the effective amount is an amount sufficient to reduce a Cellulite Severity Scale (CSS) grading of an EFP-affected site on the subject by at least one CSS grade, or to cause significant changes in total score from baseline according to the modified Hexsel CSS, or to achieve 1-level severity improvement as determined by the modified Hexsel CSS as compared to the baseline.

Preferably, the effective amount is an amount sufficient to cause significant changes in total score from baseline according to the modified Hexsel CSS.

Preferably, the effective amount is an amount sufficient to achieve 1-level severity improvement as determined by the modified Hexsel CSS as compared to the baseline.

Preferably, the amphiphilic nanoparticles are a plurality of micelles formed by the non-ionic surfactant, and the active ingredients are one or more lipophilic components. At least a portion of the lipophilic components are encapsulated in the micelles.

Preferably, the non-ionic surfactant includes at least one member selected from polysorbate 80 (Tween 80; polyoxyethylene (20) sorbitan monooleate), polyoxyl 15 hydroxystearate (Solutol® HS 15), polyoxyethylene derivatives, and polyoxyethylene castor oil derivatives.

Preferably, the polymeric carrier has a molecular weight of between 1000 and 1,000,000 g/mol, and includes at least one member selected from acrylate (e.g., alkyl-acrylate, methyl-acrylate, ethyl-acrylate, methacrylate, and ethacrylate polymer or copolymer), cellulose or cellulose derivative, polyvinyl alcohol (PVA), poly(lactic-co-glycolic acid) (PLGA), poly(L-lactide) (PLLA), poly(D-lactide) (PDLA), polyvinylpyrrolidone (PVP) or PVP-based carrier, polyethylene glycol (PEG) or PEG-based carrier, or any combination thereof.

Preferably, the lipid carrier includes at least one member selected from natural phospholipids (e.g., phosphatidylcholine (PC)), synthetic phospholipids (e.g., dipalmitoylphosphatidylcholine (DPPC), distearoyl phosphatidyl choline (DSPC), dipalmitoylethanolamine (DPPE), dioleoylphosphatidylcholine (DOPC), dioleoylphosphatidylglycerol (DOPG), phosphatidylethanolamine (PE) and sphingomyelin (SM)), and cholesterol.

Preferably, the active ingredients include one or more lipophilic components. The lipophilic components include at least one member selected from curcumin, quercetin, puerarin, oxyresveratrol, resveratrol, and derivatives, metabolites, or isomers thereof.

Preferably, the active ingredients include one or more hydrophilic components. The hydrophilic components include at least one member selected from green tea extract, epigallocatechin gallate, epicatechin, epicatechingallate, epigallocatechin, gallocatechingallate, gallocatechin, catechingallate, catechin, epigallocatechin gallate (EGCG), caffeine, carnitine, L-carnitine, synephrine, and chlorogenic acid.

Preferably, the weight ratio of the active ingredients to the non-ionic surfactant, the polymeric carrier, or the lipid carrier falls within a range of 1:2 to 1:500.

Preferably, the concentration of the active ingredients in the pharmaceutical composition falls within a range of 0.2 mg/g to 500 mg/g.

Preferably, the diameter of each of the amphiphilic nanoparticles is less than 200 nm, and the polydispersity index (PDI) of the amphiphilic nanoparticles is less than 0.4.

Preferably, the active ingredients include one or more lipophilic components and one or more hydrophilic components. The lipophilic components include at least one member selected from curcumin, quercetin, puerarin, oxyresveratrol, resveratrol, and derivatives, metabolites, or isomers thereof. The hydrophilic components include at least one member selected from green tea extract, epigallocatechin gallate, epicatechin, epicatechingallate, epigallocatechin, gallocatechingallate, gallocatechin, catechingallate, catechin, epigallocatechin gallate (EGCG), caffeine, carnitine, L-carnitine, synephrine, and chlorogenic acid.

Preferably, the weight ratio of the active ingredients to the non-ionic surfactant, the polymeric carrier, or the lipid carrier falls within a range of 1:2 to 1:500.

Preferably, the concentration of the lipophilic components in the pharmaceutical composition falls within a range of 0.2 mg/g to 500 mg/g, and the concentration of the hydrophilic components in the pharmaceutical composition falls within a range of 0.1 mg/g to 500 mg/g.

Preferably, the weight ratio of the lipophilic components to the hydrophilic components in the pharmaceutical composition falls within a range of 30:1 to 1:10.

Preferably, the pharmaceutical composition further includes at least one of member selected from a solvent, a cosolvent, a cosurfactant, a suspending agent, and an oil phase excipient.

The pharmaceutical compositions of the present disclosure exhibit a curative and preventive effect on EFP, and minimizes adverse reactions and side effects, such as necrosis of the surrounding cells and inflammation reactions. The methods of the present disclosure can be implemented by direct injection, subcutaneous implantation, intravenous injection, implanted infusion, cream, patch, or other skin delivery methods, without the need or assistance of any surgery or equipment.

### BRIEF DESCRIPTION OF THE DRAWINGS

The disclosure will become more fully understood from the detailed description given herein below for illustration only, and thus not limitative of the disclosure, wherein:

FIG. 1 shows photo images of the photonumeric descriptors of the modified Hexsel Cellulite Severity Scale (CSS) according to the prior art;

FIGS. 2A-2C are photo images showing the improvement of EFP in three human subjects after treatment by the curcumin-resveratrol complex pharmaceutical composition in accordance with an embodiment of the present disclosure;

FIGS. 3A-3B are bar charts showing the changes in total score from baseline according to the modified Hexsel CSS on week 2 and week 4, respectively, in the human subjects after treatment by the curcumin-resveratrol complex pharmaceutical composition in accordance with an embodiment of the present disclosure;

FIGS. 4A-4B are photo images showing the changes in EFP severity in subjects treated by 40 mg of the curcumin-resveratrol complex pharmaceutical composition in accordance with an embodiment of the present disclosure;

FIG. 5 are photo images showing the changes in EFP severity in a subject treated by 60 mg of the curcumin-resveratrol complex pharmaceutical composition in accordance with an embodiment of the present disclosure; and

FIG. 6 are photo images showing the changes in EFP severity in a subject treated by 80 mg of the curcumin-resveratrol complex pharmaceutical composition in accordance with an embodiment of the present disclosure.

### DETAILED DESCRIPTION OF THE PREFERRED EMBODIMENTS

In a first aspect of the present disclosure, a method for treating and/or preventing EFP in a subject in need thereof is provided. The method includes administering an effective amount of a pharmaceutical composition to the subject. The pharmaceutical composition includes a plurality of micelles and one or more lipophilic or hydrophilic components. The micelles are formed by a pharmaceutically acceptable non-ionic surfactant, the hydrophilic-lipophilic balance value (HLB value) of which is greater than 9. At least a portion of the lipophilic or hydrophilic components are encapsulated in the micelles.

The pharmaceutical composition may be prepared by the steps of: (1a) dissolving the lipophilic components in an organic solvent to form a solution; (2a) adding the non-ionic surfactant to the solution and stirring the solution to volatilize the organic solvent; (3a) after the organic solvent is completely volatilized, adding a pharmaceutically acceptable aqueous solution to obtain the pharmaceutical composition. Alternatively, the pharmaceutical composition may be prepared by the steps of: (1b) dissolving the hydrophilic components in an aqueous solvent to form a solution; (2b) adding the non-ionic surfactant to the solution; (3b) adding a pharmaceutically acceptable non-aqueous solution to obtain the pharmaceutical composition.

Specifically, in Step (1a), the lipophilic components may include, but are not limited to, at least one member selected from curcumin, quercetin, puerarin, oxyresveratrol, resveratrol, and derivatives, metabolites, or isomers thereof. In other words, the lipophilic components encapsulated in the micelles may be curcumin, quercetin, puerarin, oxyresveratrol, resveratrol, other lipophilic therapeutic agents, derivatives, metabolites, or isomers of any of the foregoing components, or any combination thereof. In a preferred embodiment, the lipophilic components include curcumin and resveratrol at a weight ratio of 4:1. The organic solvent may include, but is not limited to, hydrophobic solvents, hydrophilic solvents, other solvents having a boiling point lower than 100° C., or any combination thereof. Specifically, the hydrophobic solvents may include, but are not limit to, ethers (e.g., ether, diethyl ether), or any combination thereof; the hydrophilic solvents may include, but are not limit to, ester (e.g., ethyl acetate), alcohols (e.g., methanol, ethanol, isopropanol, ethylene glycol), dimethylformamide (DMF), ketones (e.g., acetone), formaldehyde, acetonitrile, dimethyl sulfoxide (DMSO), or any combination thereof.

In Step (1b), the hydrophilic components may include, but are not limited to, at least one member selected from green tea extract, epigallocatechin gallate, epicatechin, epicatechingallate, epigallocatechin, gallocatechingallate, gallocatechin, catechingallate, catechin, epigallocatechin gallate (EGCG), caffeine, carnitine, L-carnitine, synephrine, and chlorogenic acid. The aqueous solvent may include, but is not limited to, hydrophilic solvents, other solvents having a boiling point lower than 100° C., or any combination thereof. Specifically, the hydrophilic solvents may include, but are not limit to, ester (e.g., ethyl acetate), alcohols (e.g., methanol, ethanol, isopropanol, ethylene glycol), dimethylformamide (DMF), ketones (e.g., acetone), formaldehyde, acetonitrile, dimethyl sulfoxide (DMSO), or any combination thereof.

In Steps (2a) and (2b), the non-ionic surfactant may include, but is not limited to, at least one member selected from polysorbate 80 (Tween 80; polyoxyethylene (20) sorbitan monooleate), polyoxyl 15 hydroxystearate (Solutol® HS 15; KolliphorR HS 15), polyoxyethylene derivatives, and polyoxyethylene castor oil derivatives. In other words, the non-ionic surfactant used to form the micelles may be Tween 80, SolutolR HS 15, polyoxyethylene castor oil derivatives, other non-ionic surfactants having an HLB value of greater than 9, or any combination thereof. The polyoxyethylene castor oil derivatives may be polyoxyl 35 castor oil (PEG-35 castor oil; Kolliphor® ELP; Cremophor® ELP), polyoxyl 40 hydrogenated castor oil (CremophorR RH 40; KolliphorR RH 40), or the combination thereof.

The weight ratio of the lipophilic components to the non-ionic surfactant may fall within the range of 1:2 to 1:500, preferably 1:20 to 1:150. The concentration of the lipophilic components in the pharmaceutical composition may fall within the range of 0.2 mg/g to 500 mg/g. The diameter of each of the micelles is less than 50 nm. The polydispersity index (PDI) of the micelles is less than 0.4.

Alternatively, the weight ratio of the hydrophilic components to the non-ionic surfactant may fall within the range of 1:2 to 1:500. The concentration of the hydrophilic components in the pharmaceutical composition may fall within the range of 0.1 mg/g to 500 mg/g. The diameter of each of the micelles is less than 50 nm. The polydispersity index (PDI) of the micelles is less than 0.4.

In Step (3a), the pharmaceutically acceptable aqueous solution may be, but is not limited to, water for injection, aqueous solution for injection, or normal saline. The pharmaceutically acceptable aqueous solution may further be added with one or more local anesthetics, antioxidants, or other hydrophilic agents (e.g., green tea extract). The weight ratio of the lipophilic components over the pharmaceutically acceptable aqueous solution may fall within the range of 1:400 to 3:50.

In Step (3b), the pharmaceutically acceptable non-aqueous solution may be, but is not limited to, benzene, alcohol, ether, carbon disulfide, or acetone. The pharmaceutically acceptable non-aqueous solution may further be added with one or more local anesthetics, antioxidants, or other lipophilic agents (e.g., curcumin). The weight ratio of the lipophilic components over the pharmaceutically acceptable non-aqueous solution may fall within the range of 1:400 to 3:50.

The local anesthetics may include, but is not limited to, amides, para-aminobenzoic acid esters, amino ethers, or any combination thereof. The amides may be dibucaine, lidocaine, mepivacaine HCl, bupivacine HCl, pyrrocaine HCl, prilocaine HCl, digammacaine, oxethazaine, or any combination thereof. The para-aminobenzoic acid esters may be butacaine, dimethocaine, tutocaine, or any combination thereof. The amino ethers may be quinisocaine, pramocaine, or the combination thereof. The antioxidant may include, but is not limited to, beta-carotene, lutein, lycopene, bilirubin, vitamin A, vitamin C (ascorbic acid), vitamin E, uric acid, nitric oxide, nitroxide, pyruvate, catalase, superoxide dismutase, glutathione peroxidases, N-acetyl cysteine, naringenin, other oxidation inhibiting, or any combination thereof.

In some embodiments, the pharmaceutical composition may include one or more lipophilic components and one or more hydrophilic components. At least a portion of the lipophilic components and the hydrophilic components is encapsulated in the micelles. In these embodiments, the pharmaceutical composition may be prepared by the steps of: (1) dissolving the lipophilic components and the hydrophilic components in an organic solvent to form a solution; (2) adding the non-ionic surfactant to the solution and stirring the solution to volatilize the organic solvent; (3) after the organic solvent is completely volatilized, adding a pharmaceutically acceptable aqueous solution to obtain the pharmaceutical composition. The organic solvent may include, but is not limited to, hydrophobic solvents, hydrophilic solvents, other solvents having a boiling point lower than 100° C., or any combination thereof. Specifically, the hydrophobic solvents may include, but are not limit to, ethers (e.g., ether, diethyl ether), or any combination thereof; the hydrophilic solvents may include, but are not limit to, ester (e.g., ethyl acetate), alcohols (e.g., methanol, ethanol, isopropanol, ethylene glycol), dimethylformamide (DMF), ketones (e.g., acetone), formaldehyde, acetonitrile, dimethyl sulfoxide (DMSO), or any combination thereof.

The lipophilic components may include, but are not limited to, at least one member selected from curcumin, quercetin, puerarin, oxyresveratrol, resveratrol, and derivatives, metabolites, or isomers thereof. Preferably, the lipophilic components include curcumin and resveratrol at a weight ratio of 4:1. The weight ratio of the lipophilic components to the non-ionic surfactant may fall within the range of 1:2 to 1:500, preferably 1:20 to 1:150. The concentration of the lipophilic components in the pharmaceutical composition may fall within the range of 0.2 mg/g to 500 mg/g. The hydrophilic components may include, but are not limited to, at least one member selected from green tea extract, epigallocatechin gallate, epicatechin, epicatechingallate, epigallocatechin, gallocatechingallate, gallocatechin, catechingallate, catechin, epigallocatechin gallate (EGCG), caffeine, carnitine, L-carnitine, synephrine, and chlorogenic acid. The weight ratio of the hydrophilic components to the non-ionic surfactant may fall within the range of 1:5 to 1:500. The concentration of the hydrophilic components in the pharmaceutical composition may fall within the range of 0.1 mg/g to 500 mg/g. The weight ratio of the lipophilic components to the hydrophilic components in the pharmaceutical composition may fall within the range of 30:1 to 1:10.

In some embodiments, the pharmaceutical composition may further include at least one of member selected from a solvent, a cosolvent, a cosurfactant, a suspending agent, an oil phase excipient, and an antimicrobial agent. In other words, the pharmaceutical composition may further include a solvent, a cosolvent, a cosurfactant, a suspending agent, an oil phase excipient, an antimicrobial agent, or any combination thereof. The solvent is any substance, usually liquid, which is capable of dissolving one or several substances, thus creating a solution and/or forming micelles. The cosolvent is used to increase solubility of the components encapsulated in the micelles. The cosolvent may be, but is not limited to, polyethylene glycol, propylene glycol, ethanol, other cosolvents that can increase solubility of the lipophilic or hydrophilic components, or any combination thereof; specifically, the polyethylene glycol may be PEG 200, PEG 400, PEG 600, or any combination thereof. The cosurfactant is a chemical substance that is used in addition to a surfactant to improve its performance. In other words, cosurfactant is a second surfactant that is used in conjunction with a primary surfactant. The cosurfactant may be, but is not limit to, ethanol, propylene glycol, poly(ethylene glycol)(PEG), or any combination thereof. The suspending agent is used to reduce precipitation of the micelles or the components encapsulated therein. The suspending agent may be, but is not limited to, sodium alginate, glycerol, carboxymethylcellulose sodium, mannitol, other suspending agents that can reduce precipitation of the micelles or the lipophilic or hydrophilic components, or any combination thereof. The oil phase excipient is used to enhance stability of the pharmaceutical composition and/or solubility of the encapsulated components. The oil phase excipient may be, but is not limited to, unsaturated fatty acids, glycerol, triglycerides, other oil phase excipients that can enhance stability of the micelles and/or solubility of the lipophilic or hydrophilic components, or any combination thereof; specifically, the unsaturated fatty acids may be, but are not limited to, oleic acid, castor oil, sesame oil, cottonseed oil, soybean oil, safflower oil, corn oil, or any combination thereof; the triglycerides may be, but are not limited to, medium chain triglycerides. The antimicrobial agent is defined as a natural or synthetic substance that kills or inhibits the growth of microorganisms such as bacteria, fungi, and algae. In other words, antimicrobial agents are therapeutic substances used to prevent or treat infections. The antimicrobial agents may be, but is not limit to, antiseptics, antibiotics, antivirals, antifungals, and antiparasitics.

The pharmaceutical composition may be further formulated so that it is administered to the subject in the form of an injection, a microneedle, an implant, a transdermal patch, a cream, a lotion, a dispersion, a gel, an ointment, or a suspension.

The subject may be a mammal (e.g., human). The pharmaceutical composition may be administered to a local site (e.g., an EFP-affected site) of the subject. The edge of the local site may be defined at the discretion of the operating physician. The pharmaceutical composition may be administered subcutaneously, preferably 1-10 mm under the subcutaneous tissue.

In the case of a human subject, the pharmaceutical composition may be administered subcutaneously to the thighs (e.g., posterolateral thigh), buttocks, lower limbs, pelvic region, abdomen, or other EFP-affected sites of the human.

The pharmaceutical composition may be administered via a parenteral route, in any known non-oral forms. The parenteral route of administration may include injection, microneedle, implant, topical administration, transdermal administration, or any non-enteral routes.

In some embodiments, the pharmaceutical composition is formulated into an injectable composition and may be administered to the subject by injection, microneedle, or implant. For example, the pharmaceutical composition may be injected subcutaneously to a treatment area of a human subject. In every course of treatment, the amount of injection may be 0.01-50 mg/cm², preferably 1-2 mg/cm² of the treatment area. The distance between every two injection sites may be at least 0.5 cm. The number of injection sites may vary between different body parts.

In some embodiments, the pharmaceutical composition is formulated into a topical or transdermal composition and may be administered to the subject topically or transdermally. For example, the pharmaceutical composition may be topically applied to a treatment area of the human subject at an amount of 0.5-20 mg/cm² of the treatment area.

The subject may be subjected to at least one course of treatment. To achieve a desirable effect, the pharmaceutical composition may be administered at a frequency of once every other day, at least once every week, at least once every two weeks, at least once every month, or at least once. The total number of treatment courses is not limited herein; the treatment may continue until significant reduction of EFP is observed.

In the present disclosure, the effective amount may be an amount sufficient to achieve at least one of the following efficacy endpoints: (a) to reduce a depth of a treatment area by at least 10%; (b) to reduce a width of the treatment area by at least 10%; (c) to reduce a length of the treatment area by at least 5%; (d) to reduce an overall volume of the treatment area by at least 10%; and (e) to reduce a surface area of the treatment area by at least 10%. In some embodiments, the effective amount may be an amount sufficient to reduce a Cellulite Severity Scale (CSS) grading of an EFP-affected site on the subject by at least one CSS grade, or to cause significant changes in total score from baseline according to the modified Hexsel CSS, or to achieve 1-level severity improvement as determined by the modified Hexsel CSS as compared to the baseline.

In a second aspect of the present disclosure, another method for treating and/or preventing EFP in a subject in need thereof is provided. The method includes administering an effective amount of a pharmaceutical composition to the subject. The pharmaceutical composition includes a plurality of amphiphilic nanoparticles having one or more active ingredients encapsulated therein. Each of the amphiphilic nanoparticles is formed by a non-ionic surfactant, a polymeric carrier, or a lipid carrier. The hydrophilic-lipophilic balance (HLB) value of the non-ionic surfactant is greater than 9.

In some embodiments, the amphiphilic nanoparticles are a plurality of micelles or emulsions encapsulating the one or more active ingredients and formed by the non-ionic surfactant.

In some embodiments, the amphiphilic nanoparticles are a plurality of polymeric nanospheres or polymeric nanocapsules encapsulating the one or more active ingredients and formed by the polymeric carrier.

In some embodiments, the amphiphilic nanoparticles are a plurality of liposomes encapsulating the one or more active ingredients and formed by the lipid carrier.

In some embodiments, each of the amphiphilic nanoparticles includes a hydrophobic core and a hydrophilic envelope. The one or more active ingredients is encapsulated in the hydrophobic core, and the hydrophobic core is encapsulated in the hydrophilic envelope. The hydrophobic core is formed by hydrophobic end groups of the non-ionic surfactant, the polymeric carrier, or the lipid carrier. The hydrophilic envelope is formed by hydrophilic head groups of the non-ionic surfactant, the polymeric carrier, or the lipid carrier.

In some embodiments, each of the amphiphilic nanoparticles includes a hydrophilic core and a hydrophobic envelope. The one or more active ingredients is encapsulated in the hydrophilic core, and the hydrophilic core is encapsulated in the hydrophobic envelope. The hydrophilic core is formed by hydrophilic head groups of the non-ionic surfactant, the polymeric carrier, or the lipid carrier. The hydrophobic envelope is formed by hydrophobic end groups of the non-ionic surfactant, the polymeric carrier, or the lipid carrier.

In some embodiments, the amphiphilic nanoparticles are a plurality of micelles formed by the non-ionic surfactant, and the active ingredients are one or more lipophilic components and/or hydrophilic components.

In some embodiments, the non-ionic surfactant includes at least one member selected from polysorbate 80 (Tween 80; polyoxyethylene (20) sorbitan monooleate), polyoxyl 15 hydroxystearate (Solutol® HS 15), polyoxyethylene derivatives, and polyoxyethylene castor oil derivatives.

In some embodiments, the polymeric carrier has a molecular weight of between 1000 and 1,000,000 g/mol. The polymeric carrier includes at least one member selected from acrylate (e.g., alkyl-acrylate, methyl-acrylate, ethyl-acrylate, methacrylate, and ethacrylate polymer or copolymer), cellulose or cellulose derivative, polyvinyl alcohol (PVA), poly(lactic-co-glycolic acid) (PLGA), polyvinylpyrrolidone (PVP) or PVP-based carrier, PEG or PEG-based carrier, or any combination thereof.

In some embodiments, the lipid carrier includes at least one member selected from natural phospholipids (e.g., phosphatidylcholine (PC)), synthetic phospholipids (e.g., dipalmitoylphosphatidylcholine (DPPC), distearoyl phosphatidyl choline (DSPC), dipalmitoylethanolamine (DPPE), dioleoylphosphatidylcholine (DOPC), dioleoylphosphatidylglycerol (DOPG), phosphatidylethanolamine (PE) and sphingomyelin (SM)), and cholesterol.

The pharmaceutical composition may be prepared by the steps of: (ia) dissolving the active ingredients in an organic solvent to form a solution; (iia) adding the non-ionic surfactant to the solution and stirring the solution to volatilize the organic solvent; (iiia) after the organic solvent is completely volatilized, adding a pharmaceutically acceptable aqueous solution to obtain the pharmaceutical composition. Alternatively, the pharmaceutical composition may be prepared by the steps of: (ib) dissolving the hydrophilic components in an aqueous solvent to form a solution; (iib) adding the non-ionic surfactant to the solution and (iiib) adding a pharmaceutically acceptable non-aqueous solution to obtain the pharmaceutical composition.

Specifically, in Step (ia), the active ingredients may include one or more lipophilic components. The lipophilic components may include, but are not limited to, at least one member selected from curcumin, quercetin, puerarin, oxyresveratrol, resveratrol, and derivatives, metabolites, or isomers thereof. In other words, the lipophilic components encapsulated in the hydrophobic core of the amphiphilic nanoparticles may be curcumin, quercetin, puerarin, oxyresveratrol, resveratrol, other lipophilic therapeutic agents, derivatives, metabolites, or isomers of any of the foregoing components, or any combination thereof. In a preferred embodiment, the lipophilic components include curcumin and resveratrol at a weight ratio of 4:1. The organic solvent may include, but is not limited to, hydrophobic solvents, hydrophilic solvents, other solvents having a boiling point lower than 100° C., or any combination thereof. Specifically, the hydrophobic solvents may include, but are not limit to, ethers (e.g., ether, diethyl ether), or any combination thereof; the hydrophilic solvents may include, but are not limit to, ester (e.g., ethyl acetate), alcohols (e.g., methanol, ethanol, isopropanol, ethylene glycol), dimethylformamide (DMF), ketones (e.g., acetone), formaldehyde, acetonitrile, dimethyl sulfoxide (DMSO), or any combination thereof.

In Step (ib), the active ingredients may include one or more hydrophilic components. The hydrophilic components may include, but are not limited to, at least one member selected from green tea extract, epigallocatechin gallate, epicatechin, epicatechingallate, epigallocatechin, gallocatechingallate, gallocatechin, catechingallate, catechin, epigallocatechin gallate (EGCG), caffeine, carnitine, L-carnitine, synephrine, and chlorogenic acid. The aqueous solvent may include, but is not limited to, hydrophilic solvents, other solvents having a boiling point lower than 100° C., or any combination thereof. Specifically, the hydrophilic solvents may include, but are not limit to, ester (e.g., ethyl acetate), alcohols (e.g., methanol, ethanol, isopropanol, ethylene glycol), dimethylformamide (DMF), ketones (e.g., acetone), formaldehyde, acetonitrile, dimethyl sulfoxide (DMSO), or any combination thereof.

In Step (iia), the non-ionic surfactant may include, but is not limited to, at least one member selected from polysorbate 80 (Tween 80; polyoxyethylene (20) sorbitan monooleate), polyoxyl 15 hydroxystearate (Solutol® HS 15; KolliphorR HS 15), polyoxyethylene derivatives, and polyoxyethylene castor oil derivatives. In other words, the non-ionic surfactant used to form the micelles may be Tween 80, SolutolR HS 15, polyoxyethylene castor oil derivatives, other non-ionic surfactants having an HLB value of greater than 9, or any combination thereof. The polyoxyethylene castor oil derivatives may be polyoxyl 35 castor oil (PEG-35 castor oil; Kolliphor® ELP; Cremophor® ELP), polyoxyl 40 hydrogenated castor oil (Cremophor® RH 40; KolliphorR RH 40), or the combination thereof. The weight ratio of the lipophilic components to the non-ionic surfactant may fall within the range of 1:2 to 1:500, preferably 1:20 to 1:150. The concentration of the lipophilic components in the pharmaceutical composition may fall within the range of 0.2 mg/g to 500 mg/g. The diameter of each of the amphiphilic nanoparticles is less than 200 nm. The polydispersity index (PDI) of the amphiphilic nanoparticles is less than 0.4.

Alternatively, the weight ratio of the hydrophilic components to the non-ionic surfactant may fall within the range of 1:2 to 1:500. The concentration of the hydrophilic components in the pharmaceutical composition may fall within the range of 0.1 mg/g to 500 mg/g. The diameter of each of the micelles is less than 50 nm. The polydispersity index (PDI) of the micelles is less than 0.4.

In Step (iiia), the pharmaceutically acceptable aqueous solution may be, but is not limited to, water for injection, aqueous solution for injection, or normal saline. The pharmaceutically acceptable aqueous solution may further be added with one or more local anesthetics, antioxidants, or other hydrophilic agents (e.g., green tea extract). The weight ratio of the lipophilic components over the pharmaceutically acceptable aqueous solution may fall within the range of 1:400 to 3:50.

In Step (iiib), the pharmaceutically acceptable non-aqueous solution may be, but is not limited to, benzene, alcohol, ether, carbon disulfide, or acetone. The pharmaceutically acceptable non-aqueous solution may further be added with one or more local anesthetics, antioxidants, or other lipophilic agents (e.g., curcumin). The weight ratio of the lipophilic components over the pharmaceutically acceptable non-aqueous solution may fall within the range of 1:400 to 3:50.

The local anesthetics may include, but is not limited to, amides, para-aminobenzoic acid esters, amino ethers, or any combination thereof. The amides may be dibucaine, lidocaine, mepivacaine HCl, bupivacaine HCl, pyrrocaine HCl, prilocaine HCl, digammacaine, oxethazaine, or any combination thereof. The para-aminobenzoic acid esters may be butacaine, dimethocaine, tutocaine, or any combination thereof. The amino ethers may be quinisocaine, pramocaine, or the combination thereof. The antioxidant may include, but is not limited to, beta-carotene, lutein, lycopene, bilirubin, vitamin A, vitamin C (ascorbic acid), vitamin E, uric acid, nitric oxide, nitroxide, pyruvate, catalase, superoxide dismutase, glutathione peroxidases, N-acetyl cysteine, naringenin, other oxidation inhibiting agents, or any combination thereof.

In some embodiments, the active ingredients may include one or more lipophilic components and one or more hydrophilic components encapsulated in the amphiphilic nanoparticles. In these embodiments, the pharmaceutical composition may be prepared by the steps of: (i′) dissolving the lipophilic components and the hydrophilic components in an organic solvent to form a solution; (ii) adding the non-ionic surfactant to the solution and stirring the solution to volatilize the organic solvent; (iii) after the organic solvent is completely volatilized, adding a pharmaceutically acceptable aqueous solution to obtain the pharmaceutical composition. The organic solvent may include, but is not limited to, hydrophobic solvents, hydrophilic solvents, other solvents having a boiling point lower than 100° C., or any combination thereof. Specifically, the hydrophobic solvents may include, but are not limit to, ethers (e.g., ether, diethyl ether), or any combination thereof; the hydrophilic solvents may include, but are not limit to, ester (e.g., ethyl acetate), alcohols (e.g., methanol, ethanol, isopropanol, ethylene glycol), dimethylformamide (DMF), ketones (e.g., acetone), formaldehyde, acetonitrile, dimethyl sulfoxide (DMSO), or any combination thereof.

The lipophilic components may include, but are not limited to, at least one member selected from curcumin, quercetin, puerarin, oxyresveratrol, resveratrol, and derivatives, metabolites, or isomers thereof. Preferably, the lipophilic components include curcumin and resveratrol at a weight ratio of 4:1. The weight ratio of the lipophilic components to the non-ionic surfactant may fall within the range of 1:2 to 1:500, preferably 1:20 to 1:150. The concentration of the lipophilic components in the pharmaceutical composition may fall within the range of 0.2 mg/g to 500 mg/g. The hydrophilic components may include, but are not limited to, at least one member selected from green tea extract, epigallocatechin gallate, epicatechin, epicatechingallate, epigallocatechin, gallocatechingallate, gallocatechin, catechingallate, catechin, epigallocatechin gallate (EGCG), caffeine, carnitine, L-carnitine, synephrine, and chlorogenic acid. The weight ratio of the hydrophilic components to the non-ionic surfactant may fall within the range of 1:5 to 1:500. The concentration of the hydrophilic components in the pharmaceutical composition may fall within the range of 0.1 mg/g to 500 mg/g. The weight ratio of the lipophilic components to the hydrophilic components in the pharmaceutical composition may fall within the range of 30:1 to 1:10.

In some embodiments, the pharmaceutical composition may further include at least one of member selected from a solvent, a cosolvent, a cosurfactant, a suspending agent, an oil phase excipient, and an antimicrobial agent. In other words, the pharmaceutical composition may further include a solvent, a cosolvent, a cosurfactant, a suspending agent, an oil phase excipient, an antimicrobial agent, or any combination thereof. The solvent is any substance, usually liquid, which is capable of dissolving one or several substances, thus creating a solution and/or forming micelles. The cosolvent is used to increase solubility of the components encapsulated in the micelles. The cosolvent may be, but is not limited to, polyethylene glycol, propylene glycol, ethanol, other cosolvents that can increase solubility of the lipophilic or hydrophilic components, or any combination thereof; specifically, the polyethylene glycol may be PEG 200, PEG 400, PEG 600, or any combination thereof. The cosurfactant is a chemical substance that is used in addition to a surfactant to improve its performance. In other words, cosurfactant is a second surfactant that is used in conjunction with a primary surfactant. The cosurfactant may be, but is not limit to, ethanol, propylene glycol, poly(ethylene glycol)(PEG), or any combination thereof. The suspending agent is used to reduce precipitation of the micelles or the components encapsulated therein. The suspending agent may be, but is not limited to, sodium alginate, glycerol, carboxymethylcellulose sodium, mannitol, other suspending agents that can reduce precipitation of the micelles or the lipophilic or hydrophilic components, or any combination thereof. The oil phase excipient is used to enhance stability of the pharmaceutical composition and/or solubility of the encapsulated components. The oil phase excipient may be, but is not limited to, unsaturated fatty acids, glycerol, triglycerides, other oil phase excipients that can enhance stability of the micelles and/or solubility of the lipophilic or hydrophilic components, or any combination thereof; specifically, the unsaturated fatty acids may be, but are not limited to, oleic acid, castor oil, sesame oil, cottonseed oil, soybean oil, safflower oil, corn oil, or any combination thereof; the triglycerides may be, but are not limited to, medium chain triglycerides. The antimicrobial agent is defined as a natural or synthetic substance that kills or inhibits the growth of microorganisms such as bacteria, fungi, and algae. In other words, antimicrobial agents are therapeutic substances used to prevent or treat infections. The antimicrobial agents may be, but is not limit to, antiseptics, antibiotics, antivirals, antifungals, and antiparasitics.

The pharmaceutical composition may be further formulated so that it is administered to the subject in the form of an injection, a cream, a lotion, a dispersion, a gel, an ointment, or a suspension.

The subject may be a mammal (e.g., human). The pharmaceutical composition may be administered to a local site (e.g., an EFP-affected site) of the subject. The edge of the local site may be defined at the discretion of the operating physician. The pharmaceutical composition may be administered subcutaneously, preferably 1-10 mm under the subcutaneous tissue.

In the case of a human subject, the pharmaceutical composition may be administered subcutaneously to the thighs (e.g., posterolateral thigh), buttocks, lower limbs, pelvic region, abdomen, or other EFP-affected sites of the human.

The pharmaceutical composition may be administered via a parenteral route, in any known non-oral forms. The parenteral route of administration may include injection, microneedle, implant, topical administration, transdermal administration, or any non-enteral routes.

In some embodiments, the pharmaceutical composition is formulated into an injectable composition and may be administered to the subject by injection. For example, the pharmaceutical composition may be injected subcutaneously to a treatment area of a human subject. In every course of treatment, the amount of injection may be 0.01-50 mg/cm², preferably 0.5-2 mg/cm² of the treatment area. The distance between every two injection sites may be at least 0.5 cm. The number of injection sites may vary between different body parts.

In some embodiments, the pharmaceutical composition is formulated into a topical composition and may be administered to the subject topically. For example, the pharmaceutical composition may be topically applied to a treatment area of the human subject at an amount of 0.5-20 mg per cm² of the treatment area.

The subject may be subjected to at least one course of treatment. To achieve a desirable effect, the pharmaceutical composition may be administered at a frequency of once every other day, at least once every week, at least once every two weeks, at least once every month, or at least once. The total number of treatment courses is not limited herein; the treatment may continue until significant reduction of EFP is observed.

In the present disclosure, the effective amount may be an amount sufficient to achieve at least one of the following efficacy endpoints: (a) to reduce a depth of a treatment area by at least 10%; (b) to reduce a width of the treatment area by at least 10%; (c) to reduce a length of the treatment area by at least 5%; (d) to reduce an overall volume of the treatment area by at least 10%; and (e) to reduce a surface area of the treatment area by at least 10%. In some embodiments, the effective amount may be an amount sufficient to reduce a Cellulite Severity Scale (CSS) grading of an EFP-affected site on the subject by at least one CSS grade, or to cause significant changes in total score from baseline according to the modified Hexsel CSS, or to achieve 1-level severity improvement as determined by the modified Hexsel CSS as compared to the baseline.

### EXAMPLES

### Experiment 1: Composition Analysis of the Pharmaceutical Composition

Letting the pharmaceutical composition stand for at least 20 minutes. If the composition does not form stratification, further analyzing it by a particle analyzer.

Determining whether the pharmaceutical composition comprises micelles or amphiphilic nanoparticles by a particle size analyzer. If the particle diameter of the pharmaceutical composition, after being analyzed by a particle analyzer, is smaller than 50 nm and the PDI value is less than 0.4, the solution of the pharmaceutical composition is deemed clear and transparent when observed by the naked eye, and the light beam can be observed when the solution of the pharmaceutical composition is shined by a laser, then it indicates that the pharmaceutical composition comprises micelles or amphiphilic nanoparticles.

If micelles or amphiphilic nanoparticles are present in the pharmaceutical composition, the pharmaceutical composition is the pharmaceutical composition for treating and/or preventing EFP of the embodiments of the present disclosure.

Preferably, if the pharmaceutical composition does not form stratification and does not precipitate after standing, the pharmaceutical composition is the preferred pharmaceutical composition of the embodiments of the present disclosure.

### Experiment 2: Preparation and Characterization of the Curcumin-Resveratrol Complex Pharmaceutical Composition

Preparation of the curcumin-resveratrol complex pharmaceutical composition (i.e., pharmaceutical compositions having micelles or amphiphilic nanoparticles that encapsulate curcumin and resveratrol):

0.2 g of resveratrol, 0.8 g of curcumin, and 150-200 mL of dichloromethane were mixed together, and stirred at 150-500 rpm at room temperature until the resveratrol and curcumin dissolved completely. 40 g of ELP was added and stirred at 100-300 rpm to volatilize the dichloromethane. Once the dichloromethane was volatilized completely, normal saline for injection was slowly added to a total volume of 200 mL. The solution was mixed well to obtain a curcumin-resveratrol ELP solution. The curcumin-resveratrol ELP solution contained micelles, the total concentration of curcumin and resveratrol was 5 mg/mL, the concentration of ELP was approximately 20%, and the weight ratio of curcumin, resveratrol, and ELP was 4:1:200.

### Experiment 3: The Effect of Complex Pharmaceutical Compositions on EFP Reduction in Human Subjects

In this experiment, 3 subjects with moderate or severe edematous fibroscleotic panniculopathy (EFP) were included; there was no special restrictions on their gender and age. The included subjects have at least one EFP area at the thighs, buttocks, lower limbs, pelvic region, and/or abdomen. The local site to be treated by the pharmaceutical composition and severity of EFP thereon were determined during screening according to the cellulite severity score.

Severity of EFP were analyzed according to the modified Hexsel Cellulite Severity Scale (CSS), as described in Hexsel, D. M., Dal'forno, T., & Hexsel, C. L. (2009) A validated photonumeric cellulite severity scale. Journal of the European Academy of Dermatology and Venereology: JEADV, 23(5), 523-528.

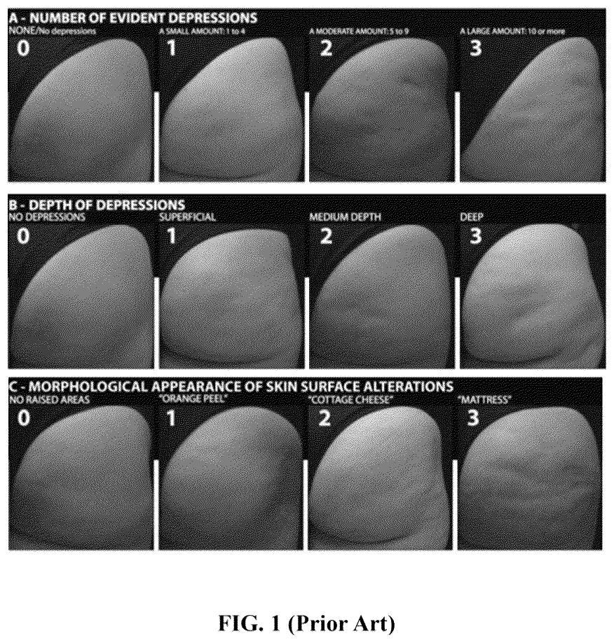

The modified Hexsel CSS is a validated comprehensive objective method of measuring EFP. As shown in FIG. 1, it is an alpha-photonumeric scale and considers three important clinical and morphological descriptors involved in EFP: (A) the number of evident depressions, (B) the depth of depressions and (C) the morphological appearance of skin surface alterations. As detailed below, each of the three indicators includes a grading of 0 to 3.

(A) Number of evident depressions: This is a score for the total number of evident depressions by visual inspection of the area to be examined. The scores are expressed as follows: 0 (zero)=No depressions; 1=A small amount (having 1 to 4 visible depressions); 2=A moderate amount (having 5 to 9 visible depressions); 3=A large amount (having 10 or more visible depressions).

(B) Depth of depressions: This evaluates the depth of depressions by visual inspection of the affected area; comparison to the photo images of CSS in FIG. 1 is recommended. The scores are expressed as follows: 0 (zero)=No depressions; 1=Superficial depressions; 2=Medium deep depressions; 3=Deep depressions.

(C) Morphological appearance of skin surface alterations: This is an assessment of the different morphological patterns of skin surface alterations; comparison with the photo images of CSS in FIG. 1 is recommended. The scores are expressed as follows: 0 (zero)=No raised areas; 1=“Orange peel” appearance; 2=“Cottage cheese” appearance; 3=“Mattress” appearance.

The total score of the three descriptors allows a final classification of EFP as mild, moderate, and severe. As shown below in Table 1, the total score of 0 (Grade 0) indicates “no EFP,” the combined scores of 1 to 3 (Grade 1) indicate “mild EFP,” 4 to 6 (Grade 2) indicate “moderate EFP,” and 7 to 9 (Grade 3) indicate “severe EFP.”

TABLE 1
EFP classification according to the modified Hexsel CSS(mHSCC)

| | Cellulite Severity | Cellulite Severity |
|---|---|---|
| Grade | Total Score | Level |
| 0 | 0 | None |
| 1 | 1 to 3 | Mild |
| 2 | 4 to 6 | Moderate |
| 3 | 7 to 9 | Severe |

Preparation of curcumin-resveratrol complex pharmaceutical composition: the curcumin-resveratrol complex pharmaceutical composition was prepared as described in Experiment 2. The total concentration of curcumin and resveratrol was 5 mg/mL, and the ratio of curcumin to resveratrol was 4:1.

The curcumin-resveratrol complex pharmaceutical composition was administered to the subjects by injection according to the following procedure:

(a) identifying the severity of EFP on one of the lateral thighs, and marking one or more treatment areas thereon by a marker pen; (b) determining and marking one or more injection spots at the treatment areas; (c) injecting the pharmaceutical composition into adipose tissues at the injection spots; the injection needle was inserted next to the markings made in Step (b) to avoid transferring the ink into the tissues; (d) applying pressure to the injection spots for 10-20 seconds to stop bleeding; (e) after bleeding stopped, injecting the pharmaceutical composition into the next injection spots until all marked spots were injected; (f) massaging the treated sites by the palm heel with a moisturizer or topical antibiotic cream for approximately 60 seconds to spread the pharmaceutical composition evenly at the treated sites; and (g) repeating the preceding steps on another lateral thigh.

In this experiment, each of the subjects was injected with 2 mg/cm², totaling 80-160 mg, of the curcumin-resveratrol complex pharmaceutical composition.

EFP severity of the subjects were assessed before the treatment and on day 14 after the treatment.

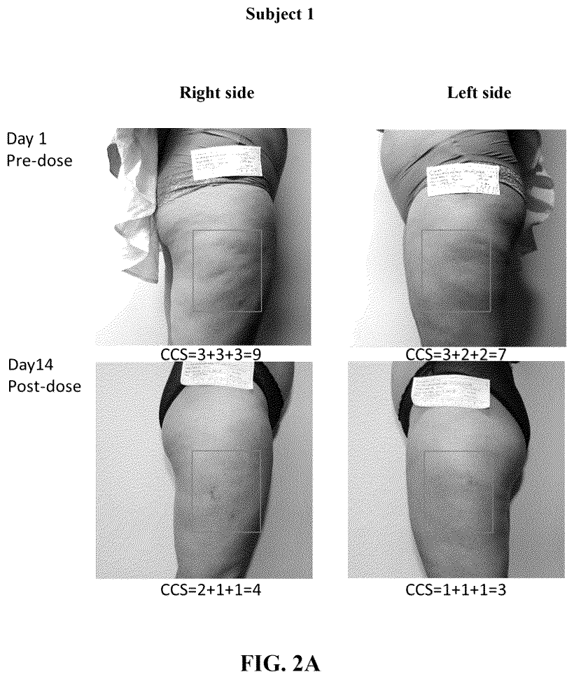

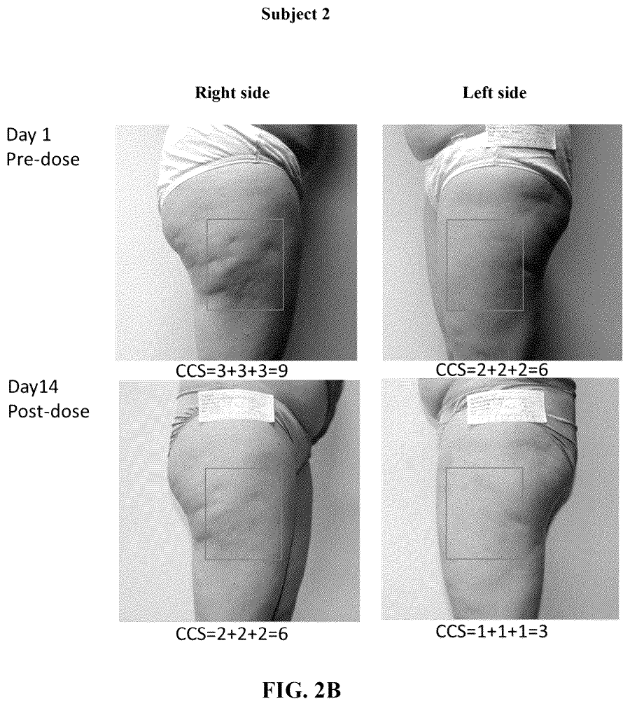

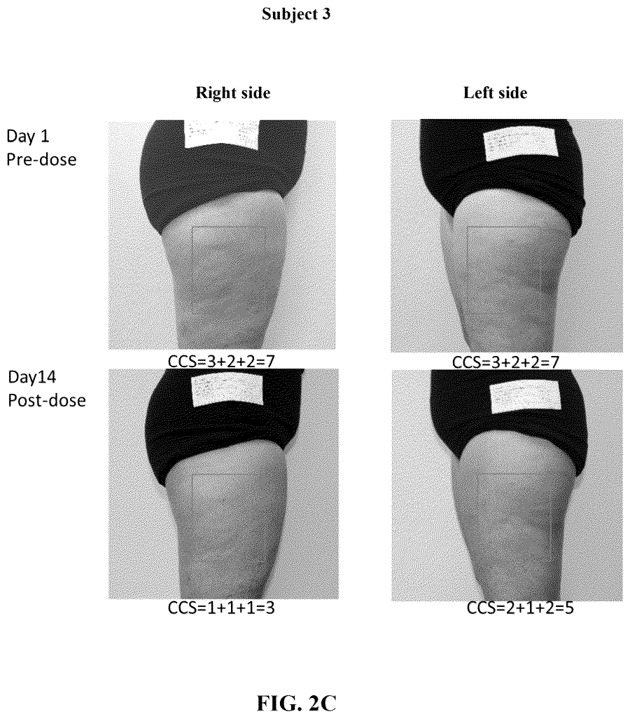

Referring to FIGS. 2A-2C. FIGS. 2A-2C are photo images showing the improvement of EFP in the human subjects after treatment by the curcumin-resveratrol complex pharmaceutical composition in accordance with an embodiment of the present disclosure.

The results in FIGS. 2A to 2C showed that the total modified Hexsel CSS (mHCSS) scores on thighs of the subjects were significantly reduced two weeks after the treatment. Specifically, total mHCSS scores of the treated sites were reduced by at least one grade. For example, as shown in FIG. 2A, cellulite severity on the right thigh of Subject 1 was downgraded from Grade 3 (total mHCSS score=9) to Grade 2 (total mHCSS score=4), and that on the left thigh of Subject 1 was downgraded from Grade 3 (total mHCSS score=7) to Grade 1 (total mHCSS score=3). Similarly, as shown in FIG. 2B, cellulite severity on the right thigh of Subject 2 was downgraded from Grade 3 (total mHCSS score=9) to Grade 2 (total mHCSS score=6), and that on the left thigh of Subject 2 was downgraded from Grade 2 (total mHCSS score=6) to Grade 1 (total mHCSS score=3). As shown in FIG. 2C, cellulite severity on the right thigh of Subject 3 was downgraded from Grade 3 (total mHCSS score=7) to Grade 1 (total mHCSS score=3), and that on the left thigh of Subject 3 was downgraded from Grade 3 (total mHCSS score=7) to Grade 2 (total mHCSS score=5).

In addition, analysis of treated sites showed that the curcumin-resveratrol complex pharmaceutical composition achieved at least one of the following efficacy endpoints: (a) depth of the treatment area was reduced by at least 10%; (b) width of the treatment area was reduced by at least 10%; (c) length of the treatment area was reduced by at least 5%; (d) overall volume of the treatment area was reduced by at least 10%; and (e) surface area of the treatment area was reduced by at least 10%.

### Experiment 4: The Effective Doses of Complex Pharmaceutical Compositions on EFP Reduction in Human Subjects

In this experiment, 12 subjects were included. The enrollment criteria were: female aged from 18 to 64 years old, having a body weight ≥50 kg, having a total score of modified Hexsel Cellulite Severity Scale (CSS) ≥4 and ≤8 on screening day and on day 1 of the experiment, having a good skin condition on treatment area, and being generally in good health (e.g., normal liver, renal, cardiovascular, coagulation, and immune functions, normal blood sugar level, and free from infectious disease and cancer). The local site to be treated by the pharmaceutical composition and severity of EFP thereon were determined on the screening day according to the modified Hexsel CSS as described in Experiment 3.

Preparation of curcumin-resveratrol complex pharmaceutical composition: the curcumin-resveratrol complex pharmaceutical composition was prepared as described in Experiment 2. The total concentration of curcumin and resveratrol was 5 mg/mL, and the ratio of curcumin to resveratrol was 4:1.

The subjects were randomly assigned into three dose groups, Groups 1-3, each having 4 subjects. On the treatment areas, Group 1 was injected with 40 mg of the curcumin-resveratrol complex pharmaceutical composition, with a unit dose of 1 mg/cm²; Group 2 was injected with 60 mg of the curcumin-resveratrol complex pharmaceutical composition, with a unit dose of 1.5 mg/cm²; and Group 3 was injected with 80 mg of the curcumin-resveratrol complex pharmaceutical composition, with a unit dose of 2 mg/cm².

The curcumin-resveratrol complex pharmaceutical composition was administered on the lateral thighs of the subjects on day 1; appearance of the lateral thighs was observed and the total score of modified Hexsel CSS was recorded on the second and fourth weeks after treatment. The pharmaceutical composition was administered according to the following procedure: (a) identifying the severity of EFP on one of the lateral thighs, and marking one or more treatment areas thereon by a marker pen; (b) determining and marking one or more injection spots at the treatment areas; (c) injecting the pharmaceutical composition into adipose tissues at the injection spots; the injection needle was inserted next to the markings made in Step (b) to avoid transferring the ink into the tissues; (d) applying pressure to the injection spots for 10-20 seconds to stop bleeding; (e) after bleeding stopped, injecting the pharmaceutical composition into the next injection spots until all marked spots were injected; (f) massaging the treated sites by the palm heel with a moisturizer or topical antibiotic cream for approximately 60 seconds to spread the pharmaceutical composition evenly at the treated sites; and (g) repeating the preceding steps for another lateral thigh.

In this experiment, the primary efficacy endpoint was: change in total scores from baseline according to the modified Hexsel CSS on week 2 and week 4 after treatment; and the secondary efficacy endpoint was: percentage of the subjects' thighs that achieved at least 1-level severity improvement as determined by the total scores of modified Hexsel CSS on week 2 and week 4 after treatment as compared to the baseline.

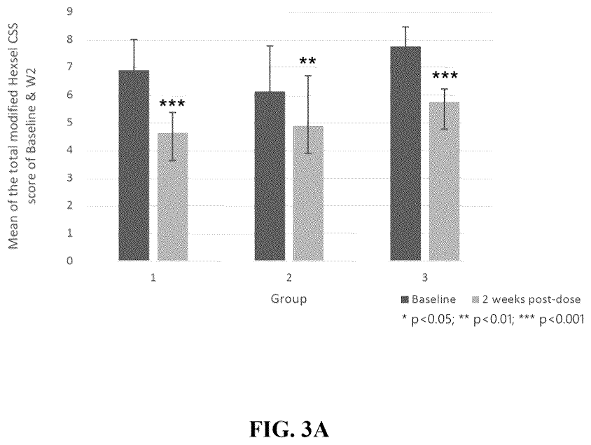

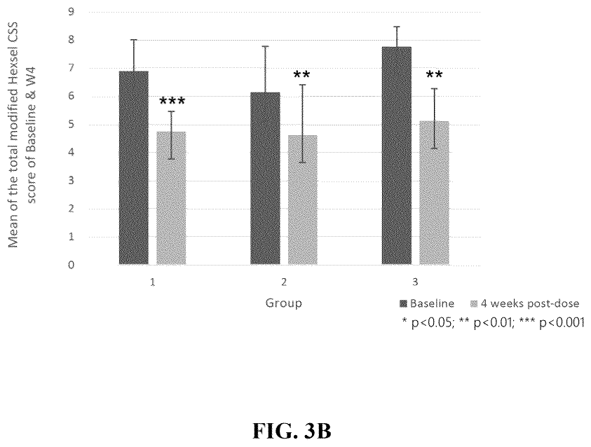

Referring to Table 2 and FIGS. 3A and 3B. In regard to the primary efficacy endpoint, the three groups exhibited significant reduction in the total score of modified Hexsel CSS from baseline on week 2 after treatment (W2), as shown in Table 2 and FIG. 3A. Similar results were also observed in week 4 after treatment (W4), as shown in Table 2 and FIG. 3B. In Table 2, n refers to the number of treated sites (e.g., two lateral thighs of four subjects=eight sites).

TABLE 2
Total scores in modified Hexsel CSS and changes thereof

| | Unit | | | | | | |
|---|---|---|---|---|---|---|---|
| | Dose | | | Total Score in Modified Hexsel CSS | | Score Change | |
| | (mg/ | | | (mean ± SD) | | (mean ± SD) | |
| Group | cm²) | n | Baseline | W 2 | W 4 | W 2 | W 4 |
| 1 | 1.0 | 8 | 6.88 ± 1.13 | 4.63 ± 0.74 | 4.75 ± 0.71 | −2.25 ± 0.89 | −2.13 ± 0.84 |
| 2 | 1.5 | 8 | 6.13 ± 1.64 | 4.88 ± 1.81 | 4.63 ± 1.77 | −1.25 ± 1.04 | −1.50 ± 0.93 |
| 3 | 2.0 | 8 | 7.75 ± 0.71 | 5.75 ± 0.5 | 5.13 ± 1.13 | −2.00 ± 0.93 | −2.63 ± 1.51 |

Referring to Table 3. In regard to the secondary efficacy endpoint, the percentage of the subjects' thighs that achieved at least 1-level severity improvement as determined by the total scores of modified Hexsel CSS on week 2 after treatment (W2) and week 4 after treatment(W4) as compared to baseline is shown in Table 3. In Groups 1 and 2, three out of the eight subjects' thighs exhibited significant improvement on week 2; the numbers further increased on week 4. In Group 3, seven out of the eight subjects' thighs exhibited significant improvement on week 2. The results demonstrate that the curcumin-resveratrol complex pharmaceutical composition can significantly reduce EFP severity in just one course of treatment.

TABLE 3
Percentage of subjects' thighs that achieved
at least 1-level severity improvement

| | | Unit Dose | | |
|---|---|---|---|---|
| Group | n | (mg/cm²) | W 2 | W 4 |
| 1 | 8 | 1.0 | 37.5% (3/8) | 50.0% (4/8) |
| 2 | 8 | 1.5 | 37.5% (3/8) | 62.5% (5/8) |
| 3 | 8 | 2.0 | 87.5% (7/8) | 87.5% (7/8) |

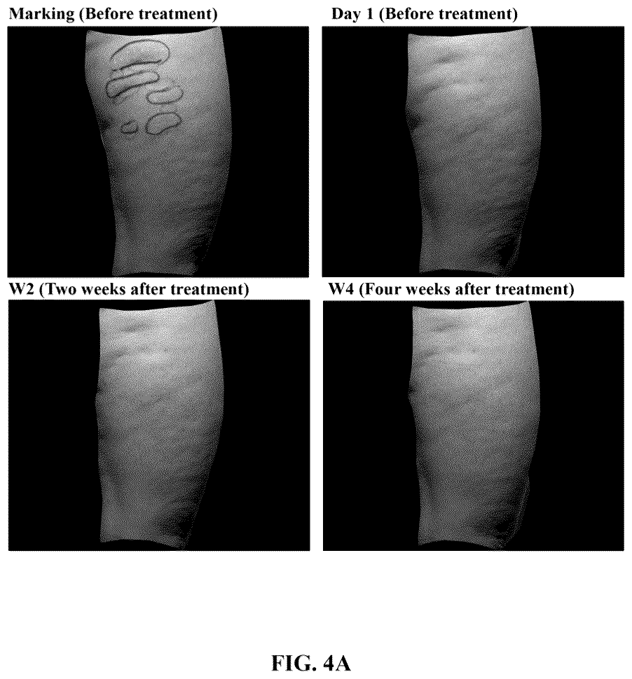

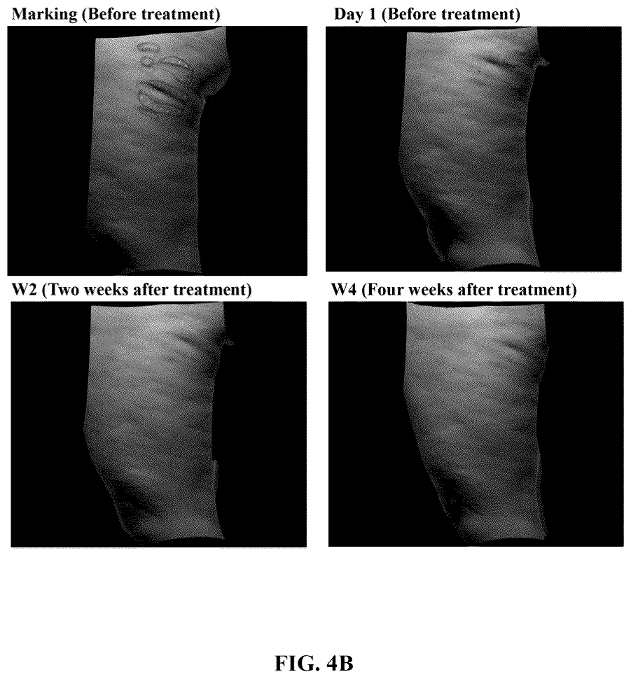

Referring to FIGS. 4A and 4B. FIGS. 4A and 4B are photo images showing the changes in EFP severity in two subjects that were representative of Group 1 (40 mg, 1.0 mg/cm²) after treatment by the curcumin-resveratrol complex pharmaceutical composition in accordance with an embodiment of the present disclosure. In the upper left image in FIG. 4A and FIG. 4B, bulges on the lateral thigh were marked by dark gray lines, depressions were marked by light gray lines, and injection spots were marked by white dots. As shown in FIGS. 4A and 4B, the total modified Hexsel CSS scores on lateral thighs of the subjects were significantly reduced on week 2 and week 4.

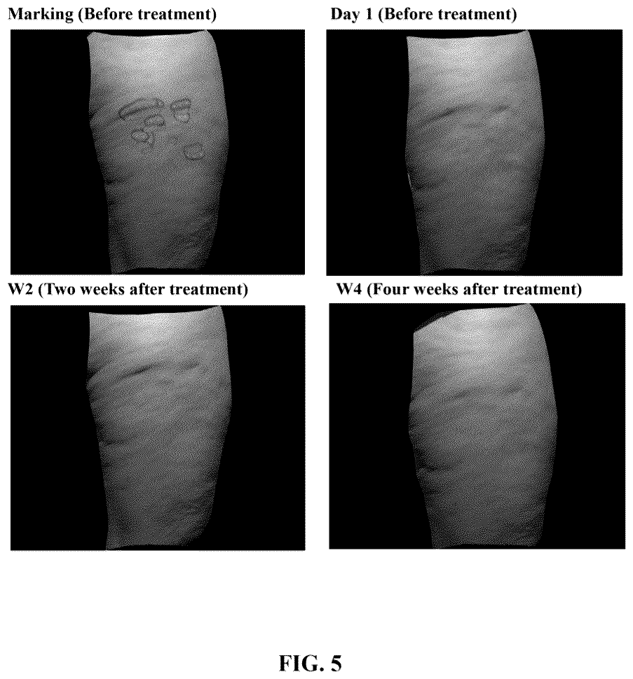

Referring to FIG. 5. FIG. 5 are photo images showing the changes in EFP severity in a subject that was representative of Group 2 (60 mg, 1.5 mg/cm²) after treatment by the curcumin-resveratrol complex pharmaceutical composition in accordance with an embodiment of the present disclosure. In the upper left image in FIG. 5, bulges on the lateral thigh were marked by dark gray lines, depressions were marked by light gray lines, and injection spots were marked by white dots. As shown in FIG. 5, the total modified Hexsel CSS scores on lateral thighs of the subjects were significantly reduced on week 2 and week 4.

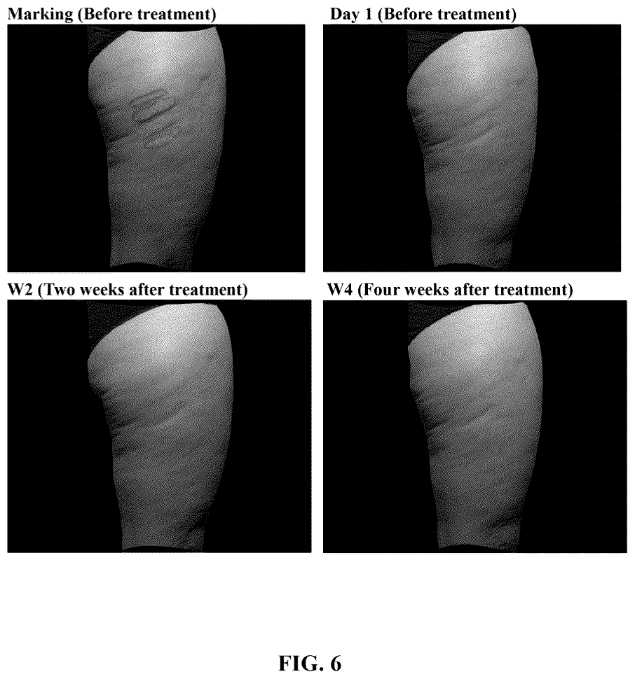

Referring to FIG. 6. FIG. 6 are photo images showing the changes in EFP severity in a subject that was representative of Group 3 (80 mg, 2.0 mg/cm²) after treatment by the curcumin-resveratrol complex pharmaceutical composition in accordance with an embodiment of the present disclosure. In the upper left image in FIG. 6, bulges on the lateral thigh were marked by dark gray lines, depressions were marked by light gray lines, and injection spots were marked by white dots. As shown in FIG. 6, the total modified Hexsel CSS scores on lateral thighs of the subjects were significantly reduced on week 2 and week 4.

### DEFINITION

In the present disclosure, the term “curcumin” refers to the curcumin extracted from natural plants or commercially available curcumin. Preferably, the purity of curcumin is 90% to 100% (wt %).

In the present disclosure, the term “resveratrol” refers to the resveratrol extracted from natural plants or commercially available resveratrol. Preferably, the purity of resveratrol is 90% to 100% (wt %).

In the present disclosure, the term “green tea extract” refers to the green tea ingredient mixture extracted by any solvent and any extraction method, commercially available green tea extract, any mixture containing at least 45% of epigallocatechin gallate (EGCG), or commercially available EGCG.

In the present disclosure, the term “non-ionic surfactant” refers to compounds having a hydrophilic end and a hydrophobic (i.e., lipophilic) end, and include polysorbate 80 (Tween 80; polyoxyethylene (20) sorbitan monooleate), polyoxyl 15 hydroxystearate (Solutola HS 15; KolliphorR HS 15), polyoxyl 35 castor oil (PEG-35 castor oil; KolliphorR ELP; Cremophor® ELP), polyoxyl 40 hydrogenated castor oil (Cremophor® RH 40; KolliphorR RH 40), other polyoxyethylene castor oil derivatives, or any combination thereof.

In the present disclosure, the term “micelles” refer to a microstructure formed by surfactants, each of which have a hydrophilic end and a hydrophobic end. The surfactants may be arranged in a way that the hydrophilic ends face outward and the hydrophobic ends face inward to form the microstructure; alternatively, the surfactants may be arranged in a way that the hydrophobic ends face outward and the hydrophilic ends face inward to form the microstructure. Preferably, the microstructure is a spherical structure, a spheroidal structure, or other microstructural structures.

In the present disclosure, the “amphiphilic nanoparticles” refer to micelles or emulsions encapsulating one or more active ingredients and formed by non-ionic surfactants; or polymeric nanospheres or polymeric nanocapsules encapsulating one or more active ingredients and formed by polymeric carriers; or liposomes encapsulating one or more active ingredients and formed by lipid carrier.

In the present disclosure, the term “state without precipitation” refers to a state wherein no observable precipitation by naked eyes.

In the present disclosure, the term “edematous fibrosclerotic panniculopathy (EFP)” can be used interchangeably with cellulite, gynoid lipodystrophy, or local lipodystrophy, all of which are associated with a topographic alteration of the skin and subcutaneous adipose commonly observed on the buttocks, lower limbs, and abdomen.

In the present disclosure, the term “effective amount” refers to a therapeutically effective amount and/or a prophetically effective amount. The therapeutically effective amount is sufficient to effectively reduce the depth, width, length, volume, or surface area of an EFP-affected site by a desired percentage, or to effectively reduce a Cellulite Severity Scale (CSS) grading of the EFP-affected site by at least one CSS grade, or to cause significant changes in total score from baseline according to the modified Hexsel CSS, or to achieve 1-level severity improvement as determined by the modified Hexsel CSS as compared to the baseline. The prophetically effective amount is sufficient to effectively prevent or delay the formation of EFP.

The term “injection” includes all type of injections, such as subcutaneous (SC) injection, intravenous (IV) injection, intraosseous injection, epidural injection, intradermal (ID) injection, or any other forms of injection.

The terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

It should be apparent to those skilled in the art that many modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “includes,” “including,” “comprises” and “comprising” should be interpreted as referring to elements, components, or steps in a non-exclusive manner, indicating that the referenced elements, components, or steps may be present, or utilized, or combined with other elements, components, or steps that are not expressly referenced.

References in the specification to “one embodiment,” “an embodiment,” “an example embodiment,” etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps.

What is claimed is:

1. A method for treating and/or preventing edematous fibrosclerotic panniculopathy (EFP) in a subject in need thereof, comprising: administering an effective amount of a pharmaceutical composition to the subject, wherein the pharmaceutical composition comprises a plurality of amphiphilic nanoparticles having one or more active ingredients encapsulated therein,
each of the amphiphilic nanoparticles is formed by of a non-ionic surfactant, a polymeric carrier, or a lipid carrier, and a hydrophilic-lipophilic balance (HLB) value of the non-ionic surfactant is greater than 9,
wherein the pharmaceutical composition is administered via a parenteral route by an injection, a microneedle, or an implant, or via topical administration or transdermal administration.

2. The method of claim 1, wherein the pharmaceutical composition is administered to thighs, buttocks, lower limbs, pelvic region, or abdomen of the subject.

3. The method of claim 1, wherein the pharmaceutical composition is administered to a treatment area of the subject by injection, and the effective unit dose injected to the treatment area is 0.01-50 mg/cm² of the treatment area.

4. The method of claim 1, wherein the pharmaceutical composition is administered to a treatment area of the subject by the microneedle or the implant, and the effective unit dose injected to the treatment area is 0.01-20 mg/cm² of the treatment area.

5. The method of claim 1, wherein the pharmaceutical composition is administered to a treatment area of the human by topical application, and the effective amount applied onto the treatment area is 0.5-20 mg/cm² of the treatment area.

6. The method of claim 1, wherein the pharmaceutical composition is administered to a treatment area of the subject by transdermal administration, and the effective amount applied onto the treatment area is 0.5-50 mg/cm² of the treatment area.

7. The method of claim 1, wherein the amphiphilic nanoparticles are a plurality of micelles formed by the non-ionic surfactant, and the active ingredients are one or more lipophilic components and/or hydrophilic components.

8. The method of claim 1, wherein the non-ionic surfactant comprises at least one member selected from polysorbate 80, polyoxyl 15 hydroxystearate, polyoxyethylene derivatives, and polyoxyethylene castor oil derivatives.

9. The method of claim 1, wherein the active ingredients comprise one or more lipophilic components, and the lipophilic components comprise at least one member selected from curcumin, quercetin, oxyresveratrol, resveratrol, and derivatives, metabolites, or isomers thereof.

10. The method of claim 1, wherein a weight ratio of the active ingredients to the non-ionic surfactant, the polymeric carrier, or the lipid carrier falls within a range of 1:2 to 1:500.

11. The method of claim 1, wherein a concentration of the active ingredients in the pharmaceutical composition falls within a range of 0.2 mg/g to 500 mg/g.

12. The method of claim 1, wherein the active ingredients comprise one or more hydrophilic components, and the hydrophilic components comprise at least one member selected from green tea extract, epigallocatechin gallate, epicatechin, epicatechingallate, epigallocatechin, gallocatechingallate, gallocatechin, catechingallate, catechin, epigallocatechin gallate (EGCG), caffeine, carnitine, L-carnitine, synephrine, and chlorogenic acid.

13. The method of claim 1, wherein a diameter of each of the amphiphilic nanoparticles is less than 200 nm, and a polydispersity index (PDI) of the amphiphilic nanoparticles is less than 0.4.

14. The method of claim 1, wherein the active ingredients comprise one or more lipophilic components and/or one or more hydrophilic components; the lipophilic components comprise at least one member selected from curcumin, quercetin, oxyresveratrol, resveratrol, and derivatives, metabolites, or isomers thereof; and the hydrophilic components comprise at least one member selected from green tea extract, epigallocatechin gallate, epicatechin, epicatechingallate, epigallocatechin, gallocatechingallate, gallocatechin, catechingallate, catechin, epigallocatechin gallate (EGCG), caffeine, carnitine, L-carnitine, synephrine, and chlorogenic acid.

15. The method of claim 14, wherein a concentration of the lipophilic components in the pharmaceutical composition falls within a range of 0.2 mg/g to 500 mg/g, and a concentration of the hydrophilic components in the pharmaceutical composition falls within a range of 0.1 mg/g to 500 mg/g.

16. The method of claim 14, wherein a weight ratio of the lipophilic components to the hydrophilic components in the pharmaceutical composition falls within a range of 30:1 to 1:10.

17. The method of claim 1, wherein the pharmaceutical composition further comprises at least one of member selected from a solvent, a cosolvent, a cosurfactant, a suspending agent, and an oil phase excipient.
